(19) United States
(12) Patent Application Publication
(10) Pub. No.: US 2006/0211637 A1
(43) Pub. Date: Sep. 21, 2006

(54) Methods of down regulating target gene expression in vivo by introduction of interfering rna

(75) Inventors: Puthupparampil V Scaria, Montgonery Village, MD (US); Martin C Woodle, Bethesda, MD (US); Patrick Y Lu, Rockville, MD (US); Qingquan Tang, Gaithersburg, MD (US); Jun Xu, Germantown, MD (US); Frank Y Xie, Germantown, MD (US)

Correspondence Address: HELLER EHRMAN WHITE & MCAULIFFE LLP, 1717 RHODE ISLAND AVE, NW, WASHINGTON, DC, 20036-3001, US

(73) Assignee: Intradigm Corporation, Rockville, MD (US)

(21) Appl. No.: 10/523,714

(22) PCT Filed: Aug. 6, 2003
(86) PCT No.: PCT/US03/24587

§ 371 Date: Mar. 28, 2006

Related U.S. Application Data
(60) Provisional application No. 60/401,029, filed on Aug. 6, 2002.

(51) Int. Cl. A61K 48/00; C12N 15/87; C08L 89/00

(52) U.S. Cl. 514/44; 435/455; 525/54.1

(57) ABSTRACT

Methods and compositions are provided for down regulation of target gene expression in vivo by RNA interference. The methods are useful for target discovery and validation of gene-based drug development, and for treatment of human diseases.

This application claims priority to U.S. provisional application Ser. No. 60/401,029, filed Aug. 6, 2002, the entirety of which is hereby incorporated by reference.

### FIELD OF THE INVENTION

The invention provide methods and compositions for down regulating target gene expression in a subject by introducing RNA interference through in vivo delivery of nucleic acid, for example, by using siRNA duplexes. The methods are useful for target discovery and validation of gene-based drug development. The invention also provides methods and compositions for clinical application of siRNA therapeutics for the treatment if disease in a subject, for example to treat cancer, infectious diseases and/or inflammatory diseases.

### BACKGROUND OF THE INVENTION

RNA interference (RNAi) is a post-transcriptional process where a double stranded RNA inhibits gene expression in a sequence specific fashion. The RNAi process occurs in at least two steps: During the first step, a longer dsRNA is cleaved by an endogenous ribonuclease into shorter, 21- or 23-nucleotide-long dsRNAs, termed “small interfering RNAs” or siRNAs. In the second step, the smaller siRNAs then mediate the degradation of a target mRNA molecule. This RNAi effect can be achieved by introduction of either longer double-stranded RNA (dsRNA) or shorter small interfering RNA (siRNA) to the target sequence within cells. Recently, it was demonstrated that RNAi can also be achieved by introducing of plasmid that generate dsRNA complementary to target gene.

RNAi methods have been successfully used in gene function determination experiments in Drosophila^{(20,22,23,25)}, C. elegans^{(14,15,16)}, and Zebrafish⁽²⁰⁾. In those model organisms, it has been reported that both the chemically synthesized shorter siRNA or in vitro transcribed longer dsRNA can effectively inhibit target gene expression. Methods have been reported that successfully achieved RNAi effects in non human mammalian and human cell cultures⁽³⁹⁻⁵⁶⁾. However, RNAi effects haveb been difficult to observe in adult animal models⁽⁵⁷⁾. This is for at least two reasons: first, introduction of a long double-stranded RNA into mammalian cells triggers an antiviral response through up-regulation of interferon gene expression, resulting in apoptosis and death of the cells, and; second, the efficiency of dsRNA delivery into the cell is too low, especially in animal disease models. Although RNAi has potential applications in both gene target validation and nucleic acid therapeutics, progress of the technology has been hindered due to the poor delivery of RNAi molecules into animal disease models. It is apparent, therefore, that improved methods for delivering RNAi molecules in vivo are greatly to be desired.

### SUMMARY OF THE INVENTION

It is therefore an object of the invention to provide methods for inhibiting expression of one or more specific genes in a mammal.

It is a further object of the invention to provide methods for treating disease in a mammal by inhibiting expression of one or more specific genes in the mammal.

In achieving these objects there has been provided a method for down regulating a pre-selected endogenous gene in a mammal, comprising administering to a tissue of the mammal a composition comprising a double-stranded RNA molecule where the RNA molecule specifically reduces or inhibits expression of the endogenous gene. This down regulation of an endogenous gene may be used for treating a disease in the mammal that is caused or exacerbated by expression of the gene. The mammal may be a human.

There also has been provided a method for treating a disease in a mammal associated with undesirable expression of a preselected endogenous gene, comprising applying a nucleic acid composition to a tissue of the mammal and substantially contemporaneously applying a pulsed electric field to the tissue, where the nucleic acid composition may be capable of reducing expression of the endogenous gene in the tissue. The disease may be cancer or a precancerous growth and the tissue may be, for example, a breast tissue, colon tissue, a prostate tissue, a lung tissue or an ovarian tissue.

The RNA molecule may be a small interfering RNA or a long double stranded RNA. The small interfering RNA molecule may have a length of about 21-23 bp. The long double stranded RNA may have a length of about 100-800 bp. The RNA may have a length of about one hundred base pairs or less.

The composition may be administered directly to a tissue of the mammal, for example via injection into a tumor or joint in the mammal.

The composition may further comprises a polymeric carrier that enhances delivery of the RNA molecule to the tissue of the mammal. The polymeric carrier may comprise a cationic polymer that binds to the RNA molecule. The cationic polymer may be an amino acid copolymer, containing, for example, histidine and lysine residues. The polymer may be a branched polymer.

The composition may contain a targeted synthetic vector that enhances delivery of the RNA molecule to the tissue of the mammal. The synthetic vector may comprise a cationic polymer, a hydrophilic polymer, and a targeting ligand. The polymer may be a polyethyleneimine, the hydrophilic polymer may be a polyethyleneglycol, and/or the targeting ligand may be a peptide comprising an RGD sequence.

In any of these methods, a pulsed electric field may be applied to the tissue substantially contemporaneously with the composition. A second electric pulse may be applied substantially contemporaneously to the tissue to enhance delivery.

The endogenous gene may be a mutated endogenous gene, and at least one mutation in the mutated gene may be in a coding or regulatory region of the gene.

The composition may be a vector composition where the vector encodes an RNA transcript operatively coupled to a regulatory sequence that controls transcription of the transcript, and where the transcript can form a double stranded RNA molecule in the tissue that specifically reduces or inhibits expression of the endogenous gene. The vector may be a viral vector or a plasmid, cosmid or bacteriophage vector. The regulatory sequence may comprise a promoter, for example a a tissue-selective promoter such as a skin-selective promoter or a tumor selective promoter. The may be selected from the group consisting of CMV, RSV LTR, MPSV LTR, SV4 AFP, ALA, OC and keratin specific promoters.

In any of these methods, the endogenous gene may be selected from the group consisting of cancer causing genes, growth factor genes, angiogenesis factor genes, protease genes, protein serine/threonine kinase genes, protein tyrosine kinase genes, protein serine/threonine phosphatase genes, protein tyrosine phosphatase genes, receptor genes, matrix protein genes, cytokine genes, growth hormone genes, and transcription factor genes. The gene may be selected from the group consisting of VEGF, VEGF-R, VEGF-R2, VEGF121, VEGF165, VEGF189, and VEGF206.

In methods involving application of an electric pulse the pulse may comprise a square wave pulse of at least 50 V that may be applied to the tissue for between about 10 and about 20 minutes The pulse may be monopolar, bipolar or of multiple polarity. The pulse may comprise an exponential decay pulse of 120 V that may be applied to the tissue for between about 10 and about 20 minutes. In each of these methods the electric pulse may be applied via an electrode selected from the group consisting of a caliper electrode, a meander electrode, a needle electrode, a micro needle array electrode, a micropatch electrode, a ring electrode, and combinations thereof. The caliper electrode may have an area of about 1 cm². The caliper electrode may be applied to a skin fold having a thickness of about 1 mm to about 6 mm.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description

### BRIEF DESCRIPTION OF THE DRAWINGS

FIG. 1 shows electroporation mediated RNAi delivery in animal disease model. Step I: local delivery of naked plasmid DNA expressing double stranded RNA in host tissue with a saline solution, of double stranded RNA (large fragment-700 bp, or 21-23 nt oligos), and both; Step II: pulsed electrical field treatment with appropriate apparatus and probes; Step III: Biological readout to detect the efficiency of RNAi inhibition of targeted protein and therapeutic efficacy.

FIG. 2 shows RNAi mediated inhibition of Luciferase Expression in a Xenograft tumor model. Luciferase expression vector (pCI-Luc) was co-delivered with specific dsRNA (Luc-dsRNA) and non-specific dsRNA (LacZ-dsRNA) at 3 concentrations intra-tumor directly. At 0.5 μg, Luciferase expression was significantly inhibited by vector expressed specific dsRNA, but not by LacZ-dsRNA. When concentrations of both specific and non-specific dsRNAs reach to 5 μg dose, the inhibition become non-specific.

FIG. 3 shows down regulation of angiogenesis factor VEGF results in inhibition of tumor growth by electroporation mediated VEGF specific RNAi delivery. It becomes a very aggressive tumor line when MCF7 transduced with VEGF165 permanently. Two times electroporation with 10 μg RNAi molecules each delayed the tumor growth.

FIG. 4 shows different inhibition dynamics with siRNA or dsRNA Although the same parameters of electroporation, the same routes of delivery and the same amount of each form of RNAi was applied, the inhibition of the tumor growth differed. DsRNA demonstrated an early strong effect verses a delayed effect mediated by siRNA. Comparing to LacZ RNAi, both dsRNA and siRNA specific to VEGF clearly demonstrated sequence specific inhibition.

FIG. 5 shows VEGFR2 specific inhibition of tumor growth. Mouse VEGFR2 gene has been considered to play a pivotal role in tumor angiogenesis and in stromal cross-talking with tumor cells. After two deliveries intratumorally of mVEGFR2 specific RNAi followed by electroporations, tumor growth clearly was delayed compared to deliveries of Luc expressing plasmid and non-specific RNAi.

FIG. 6. When siRNA duplexes (fluorescent labeled) were delivered intratumorally with electroporation enhancement, they were evenly distributed through out the tumor. This result indicated the siRNA delivery is different from plasmid which usually only localized in a small area in the tumor.

FIG. 7 shows LacZ-specific siRNA delivery into a tumor formed by MCF-7/VEGF165 cells, which has been engineered to endogenously express LacZ. The results show that 20 μg of siRNA achieved >70 percent reduction in β-Gal expression 24 hours after delivery of LacZ-siRNA.

FIG. 8. shows immunohistochemical staining of tumor tissue treated with VEGF-siRNA and LacZ-siRNA. H&E staining demonstrated a significantly different image of VEGF-siRNA treated tumor from LacZ-siRNA treated tumor (A-B). VEGF staining (C) was lost when tumor was treated with VEGF-siRNA (D). Apoptosis activity was significant upregulated in the VEGF-siRNA treated tumor.

FIG. 9. VEGF-siRNA knockdown VEGF expression at mRNA level in vitro (left panel) resulted in MDA-MB435 breast tumor growth inhibition.

FIG. 10. Co-delivery of Luciferase expression plasmid and Luc-siRNA into the MDA-MB435 tumor, demonstrated that Luc-siRNA achieved significant knockdown of luciferase expression.

FIG. 11. When VEGF-siRNA was delivered into a tumor model, the mRNA level of VEGF in the tumor tissue was down regulated.

FIG. 12. When ICT1031 or April gene expression was subject to RNAi knockdown, tumor growth was inhibited. A cell-based assay did not show significant change of apoptosis activity.

FIG. 13. When ICT1027 or GRB2 gene expression was subject to RNAi knockdown, tumor growth was inhibited and cell apoptosis activity was significant increased.

FIG. 14. When ICT1024 or EGF-AP gene expression was subject to RNAi knockdown, tumor growth was inhibited and cell apoptosis activity was significant increased.

FIG. 15. When ICT1030 gene expression was subject to RNAi knockdown, tumor growth was accelerated.

FIG. 16. PolyTran (HK polymer) carrier mediated ICT1003-siRNA delivery resulted in tumor inhibition compared to the GFP-siRNA treated tumor.

FIG. 17. Co-delivery of luciferase expression plasmid with luc-siRNA into mouse airway through a method called oraltracheal delivery, resulted in a siRNA-mediated luciferase inhibition in mouse lung. The luciferase activities from different samples were measured by harvesting the lungs first and then testing in a luminometer.

FIG. 18. Co-delivery of luciferase expression plasmid with luc-siRNA into mouse muscle through electroporation resulted in siRNA-mediated luciferase inhibition in mouse leg muscle.

FIG. 19. Co-delivery of luciferase expression plasmid with luc-siRNA into mouse joints through electroporation resulted in a siRNA-mediated luciferase inhibition in mouse leg joints.

FIG. 20. A systemic approach for siRNA delivery through IV injection showed tumor targeting effect. Tumor tissue is marked by two circles.

FIG. 21. Use of mouse VEGF a. mVEGFR1 and mVEGFR2 specific siRNA duplexes, through an IV systemic delivery, significantly decreased the neovasculature area of the front of eyes.

FIG. 22. Using the same method as in FIG. 21, the decrease of the red neovasculature in the RNAi-treated group clearly is greater than in the control group.

### DETAILED DESCRIPTION

Methods for efficient RNAi delivery in vivo are provided. In one embodiment, RNAi is delivered into a subject, for example, a human or other animal, both locally and systemically through use of a pulsed electrical field (electroporation). The methods may be used with (1) all forms of RNAi, e.g. siRNA, dsRNA and DNA-RNA duplex; (2) all forms of RNAi payloads, eg. synthetic, in vitro transcribed and vector expressed RNAi; and (3) all types of tissues and organs that are accessible for electroporation. In other methods the RNAi is delivered using a polymer carrier and via intravenous (IV) delivery.

The invention also provides the medium used for delivery of RNAi;) routes chosen for effective delivery and parameters suitable for use for in vivo electroporation. The methods of the present invention have been used to achieve down regulation of a reporter gene that is co-delivered with its corresponding RNAi. The methods also have been successfully used to demonstrate antitumor efficacy after delivery of different payload forms of the corresponding RNAi by, for example, down regulation of expression of angiogenesis factors, e.g. VEGF and VEGFR2.

The present inventors have identified certain properties of different forms of RNAi, e.g. small interfering RNA (21-23 nt) and double stranded RNA (about 700 bp) in animal disease models. This invention provided a powerful tool to achieve RNAi effects in vivo, and hold tremendous potential for various applications in functional genomic research and in nucleic acid therapeutics.

The use of RNA interference (RNAi) has been developing rapidly in cell culture and in model organisms such as Drosophila, C. elegans, and zebrafish. Studies of RNAi have found that long dsRNA is processed by Dicer, a cellular ribonuclease III, to generate duplexes of about 21 nt with 3′-overhangs, called short interfering RNA (siRNA), which mediates sequence-specific mRNA degradation (5). An understanding of the mechanisms of RNAi and its rapidly expanding application represent a major breakthrough during the last decade in the field of biomedicine. Use of siRNA duplexes to interfere with expression of a specific gene requires knowledge of target accessibility, effective delivery of the siRNA into the target cells and, for some biological applications, long-term activity of the siRNA in the cell.

Together with the rapidly growing literature on siRNA as a functional genomic tool, there is emerging interest in using siRNA molecules as novel therapeutics. Successful therapeutic applications will depend upon successful development of optimized local and systemic delivery methods. The advantages of using siRNA as a therapeutic agent are due to its specificity (3, 4), stability (18) and mechanism of action (5, 6).

In cancer, the tumorigenesis process is thought to be the result of abnormal over-expression of oncogenes, angiogenesis factors, growth factors, and mutant tumor suppressors, even though under-expression of other proteins also plays a critical role. Increasing evidences supports the notion that siRNA duplexes ar able to “knockdown” tumorigenic genes both in vitro and in vivo, resulting in significant antitumor effects (6). The present inventors have demonstrated substantial knockdown of human VEGF in MCF-7 cell, MDA-MB435 cell and 1483 cell induced xenograft tumor models, achieving tumor growth inhibition of 40-80%. It is anticipated that VEGF-siRNA induced antiangiogenesis effect will alter the microvasculature in tumor and will result in activation of tumor cell apoptosis and, on the other hand, will also enhance efficacy of the cytotoxic chemotherapeutic drugs. However, to achieve significantly improved antitumor efficacy of antiangiogenesis agents and chemotherapeutic drugs, a highly effective delivery method is necessary so that elevated concentrations of the drugs accumulate in the local tumor tissue.

The present inventors previously have described a method of validating drug targets that determines which targets controlling tumor disease and thus justify anti-tumor drug discovery (see PCT/US02/31554). This method validates targets directly in animal tumor models through transgene over-expression and eliminates targets lacking disease control. The method reduces the need for protein generation, antibodies, and/or transgenic animals, while providing clear and definitive evidence that targets actually control the disease. Moreover, the method provides valuable information that may be lost with methods that rely solely on cell-culture and miss the complex interactions of multiple cell types that result in disease pathology.

The present inventors also have described gene delivery technologies suitable for high throughput delivery into animal tumor models. See WO01/47496, the contents of which are hereby incorporated by reference in their entirety. These methods enable direct tumor administration of plasmids and achieve a significant (for example, seven-fold) increase in efficiency compared to “gold standard” nucleotide delivery reagents. Accordingly, the methods provide strong tumor expression and activity of candidate target proteins in the tumor.

This platform is a powerful tool for validation of genes that are under-expressed in tumor tissue. However, a method to achieve gene silencing is highly desired for validation of genes that are over-expressed in tumor tissue. Recently, double stranded RNA has been demonstrated to induce gene-specific silencing by a phenomenon called RNA interference (RNAi). Although the mechanism of RNAi is still not completely understood, early results suggested that this RNAi effect may be achieved in vitro in various cell types including mammalian cells.

A double stranded RNA targeted against a target mRNA results in the degradation of the target, thereby causing the silencing of the corresponding gene. Large double stranded RNA is cleaved into smaller fragments of 21-23 nucleotides long by a RNase Im like activity involving an enzyme Dicer. These shorter fragments known as siRNA (small interfering RNA) are believed to mediate the cleavage of mRNA. Although gene down regulation by RNAi mechanism has been studied in C. elegans and other lower organisms in recent past, its effectiveness in mammalian cells in culture has only recently been demonstrated. An RNAi effect recently was demonstrated in mouse using the firefly luciferase gene reporter system (57).

To develop an RNAi technology platform for in vivo gene function validation and for potential clinical application of nucleic acid therapeutics to treat human diseases, the present inventors performed several in vivo studies in tumor-bearing mouse models. In those experiments, either siRNA or dsRNA targeting a tumor related ligand (human VEGF) or receptor (mouse VEGFR2) was intratumorally delivered to nude mice bearing xenografted human MCF-7 derived tumor or human MDA-MBA35 tumors. For the first time we were able to demonstrate that RNAi can effectively silencing target gene in tumor cells in vivo and that, as a result, tumor growth was inhibited.

The present invention, thus generally described, will be understood more readily by reference to the following examples, which are provided by way of illustration and are not intended to be limiting of the present invention

### EXAMPLE 1

### Luciferase Reporter Gene Silencing in Xenografted Tumors Mediated by Co-Transfected dsRNA

To investigate whether interfering RNAs inhibit gene expression in a mouse tumor model, we used direct intratumoral injection followed by electroporation to co-deliver naked dsRNA and Luciferase expression plasmid DNA into human MDA-MB435 tumor xenografted in nude mice. Briefly, a 700 bp DNA fragment derived from the firefly Luciferase gene was PCR amplified and a T7 promoter sequence was added to both ends of the DNA fragment during the PCR reaction. The DNA fragment was then used as a DNA template for in vitro transcription. The in vitro transcription was carried out using an dsRNA generation kit from New England BioLab following the manufacturer's instructions. Two μg of luciferase expression plasmid, pCILuc, was mixed with 0.5, 2, and 5 μg dsRNA derived from Luciferase gene or LacZ-gene in a final volume of 30 μl physiologic saline. The DNA/dsRNA mixture in saline solution was directly injected into human MDA-MB-435 tumor xenografted in Ncr Nu/Nu mice with a precision injector (Stepper, Tridake).

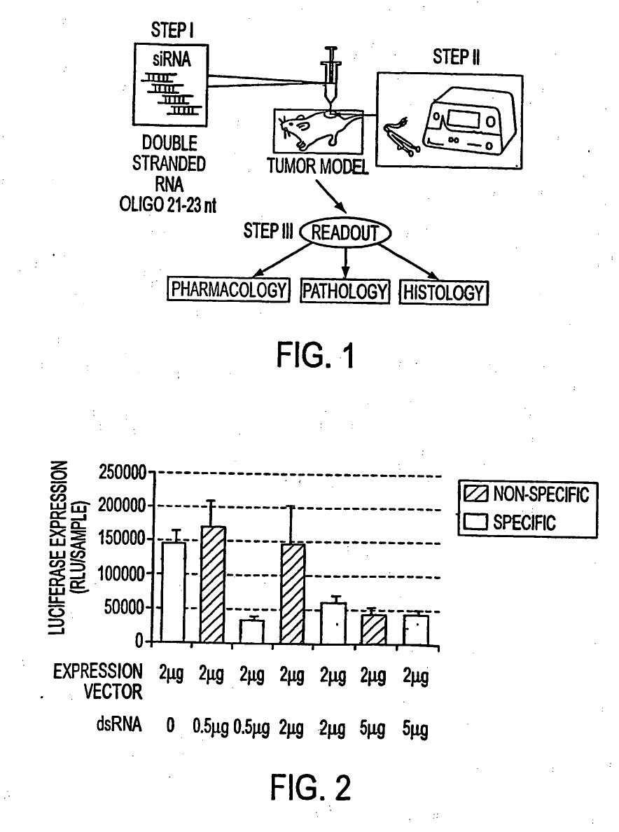

Immediately after injection, a procedure of pulsed electrical field was carried out (FIG. 1). A thin layer of conductive gel (KY Jelly) was applied to the tumor surface to ensure good contact between the plate electrodes and tumor, and electric pulses were delivered through two external plate electrodes placed at each sides of tumor using an electroporator (BIX ECM 830, San Diego). The parameters for electroporation were as follows: voltage to electrode distance ratio (Electric-Field Strength) was 200-V/cm; duration of each pulse was 20 ms; Interval time between two pulses was 1 second (1 Hz). The number of pulses was 6. Twenty-four hours post DNA injection, tumors were excised after the animals being sacrificed. Each tumor was homogenized in 800 μl of 1× lysis buffer (Promega) in a homogenizing tube (Lysing Matrix D, Q-BIOgene) using a Fastprep (Q-BIOgene) with speed at 4 for 40 seconds at 4° C. The homogenates were centrifuged at 14,000 rpm for 2 minutes after incubation on ice for 30 minutes. The supernatant was transferred into a fresh tube and 10 μl was used for luciferase activity assay using the Luciferase assay kit (Promega) and a Luminometer (Monolight 2010, Analytic Luminescence Lab.).

As illustrated in FIG. 2, the co-delivered dsRNA derived from Luciferase gene was able to silence Luciferase expression in a xenografted tumor. As little as 0.5 μg dsRNA was enough to achieve significant gene silencing against 2 μg of co-delivered pCILuc plasmid DNA. Non-specific dsRNA interference effect was observed when 5 μg dsRNA derived from LacZ gene was co-delivered with 2 μg of pCILuc plasmid DNA. No non-specific effect was observed at lower doses of dsRNA (0.5 μg and 2 ug). This is the first observation of dsRNA mediated specific gene silencing in xenografted tumor in adult mice.

### EXAMPLE 2

### RNAi Mediated Human VEGF Gene Silencing Inhibits Human MCF-7 Derivate Tumor Growth in Mice

An in vivo study was carried out to demonstrate that the introduced siRNA can not only silence the co-delivered reporter gene, but also down regulate expression of an endogenous gene, e.g. VEGF. When the target gene is a tumor control gene, down regulation of the gene causes a therapeutic efficacy: inhibition of tumor growth. Human VEGF induces angiogenesis and endothelial cell proliferation and plays an important role in regulating vasculogenesis. There are several splice variants of human VEGF including VEGF121, VEGF165, VEGF189, and VEGF206, each one comprising a specific exon addition. VEGF165 is the most predominant protein, though the transcript of VEGF121 may be more abundant VEGF165 is a heparin-binding glycoprotein that is secreted as a homodimer of 45 kDa. Most types of cells, but usually not endothelial cells themselves, secrete VEGF. Since the first-discovered VEGF, VEGF165, increases vascular permeability, it is also known as vascular permeability factor. In addition, VEGF causes vasodilatation, partly through stimulation of nitric oxide synthase in endothelial cells. VEGF also can stimulate cell migration and inhibit apoptosis.

Two animal models were used for a comparative study. The first tumor model was established with a MCF-7 breast tumor line, and the second tumor model was established with a MCF-7 derived tumor cell line, MCF-7/VEGF165. Before injection of any type of RNAi, we observed a much more aggressive tumor growth for MCF-7/VEGF165 induced tumor than that induced by MCF-7 itself This behavior has been reported and represents the role of VEGF165 as a tumor growth enhancer through an angiogenesis promoting activity. To achieve a VEGF specific down regulation, 10 μg of either siRNA (21 nt) derived from hVEGF gene or siRNA derived from LacZ gene was directly injected into xenografted MCF-7/VEGF165 tumor that over-expressing human VEGF165 in nude mice. Two siRNA (21 nt) sequences were designed to target human VEGF165 gene. VEGF_{RNAi}A sequence is 5′-ucgagacccugguggacauuu-3′ and VEGF_{RNAi}B sequence is 5′-ggccagcacauaggagagauu-3′. Both siRNAs were double-stranded with two UU overhang on both ends. For intratumoral injection, 5 μg of each of the two siRNAs makes up 10 μg of the VEGF specific siRNAs. In addition, the same amount of dsRNA (10 μg) targeting VEGF165 gene was also introduced by the same delivery method. Electric pulses were applied to tumor immediately after siRNA injection as described above. A second siRNA administration was performed on day 7 post first RNAi administration. The tumor volume was measured as an indication of hVEGF gene silencing.

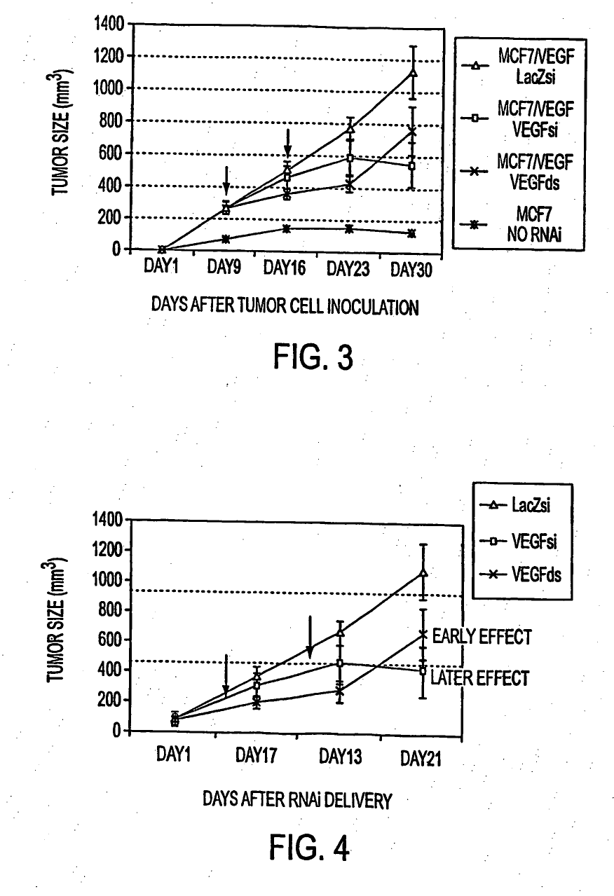

As demonstrated in FIG. 3, MCF-7/VEGF165 induced tumors treated with non-specific LacZ siRNA grew much faster than MCF-7 induced tumors. Administration of VEGF specific siRNA and dsRNA clearly demonstrated tumor growth inhibition effect Two RNAi administrations at day 9 and day 16 achieved in vivo inhibition of tumor growth. Interestingly, treatments with VEGF specific siRNA and dsRNA yielded different inhibition patterns. Treatment with VEGF siRNAs demonstrated a delayed effect which shown stronger inhibition after day 23. On the other hand, treatment with VEGF dsRNA presented an earlier inhibition even after the first administration (FIG. 4). This demonstrated that hVEGF siRNAs and hVEGF dsRNA specifically silenced the hVEGF gene in the treated tumors and therefore slowed tumor growth through the anti-angiogenesis mechanism.

### EXAMPLE 3

### RNAi Mediated Mouse VEGFR2 Gene Silencing Inhibits Human MDA-MB-435 Tumor Growth in Mice

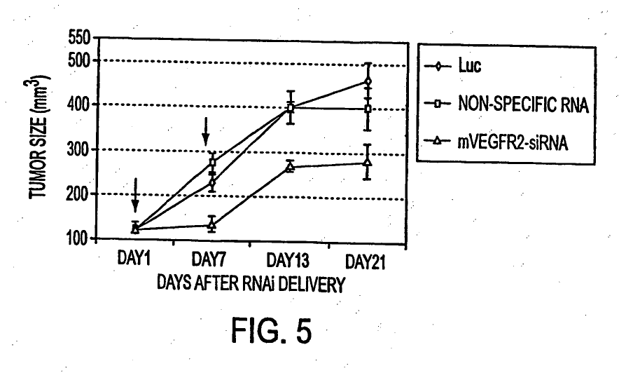

To illustrate the power of RNAi mediated gene silencing in effecting tumor growth by targeting endogenous tumor control gene, one in vivo study was carried out to silence mouse VEGFR2 gene in MCF-7 derivative tumor bearing nude mice. Two siRNAi were designed to target mouse VEGFR2 gene. VEGFR2_{RNAi}A sequence is 5′-gcucagcacacagaaagacuu-3′ and VGFR2_{RNAi} B sequence is 5′-ugcggcgguggugacaguauu-3′. Both siRNAs were double-stranded with two UU overhang on both ends. Five μg of each siRNA makes up 10 μg for each delivery. Ten μg of siRNAi derived from mVEGFR2 or LacZ gene, or 10 μg of pCILuc plasmid DNA, was directly injected into . xenografted human MCF-7 derived tumor in nude mice. Electric pulses were applied to tumor immediately after siRNAs/DNA injection as described above. A second siRNAs/DNA administration was performed on day 7 post first administration. The tumor volume was measured as an indication of mVEGFR2 gene silencing. As demonstrated in FIG. 5, tumors treated with siRNAs derived from mVEGFR2 gene grown significantly slower compared to tumors treated with pCILuc plasmid DNA or sDRNAs derived from LacZ gene. LacZ siRNAs treatment did not inhibit tumor growth, therefore demonstrating that mVEGFR2 siRNAs specifically silenced mVEGFR2 gene in treated tumor and thus slow down tumor growth rate.

To further illustrate the power of RNAi mediated gene silencing in affecting tumor growth by a targeting tumor control gene, another in vivo study was carried out to silence the mouse VEGFR2 gene in human MDA-MB435 tumor-bearing nude mice. In addition to siRNAs derived from mVEGFR2 decribed above, 10 μg of either dsRNA (700 nt in length) derived from mouse VEGFR2 gene or siRNAs derived from LacZ gene was directly injected into human MDA-MB435 xengrafted tumor in nude mice. Electric pulses were applied to tumor immediately after dsRNA/siRNAs injection as described above. A second dsRNA/siRNAi administration was performed on day 3 post first administration. Ten μg of a DNAzyme specifically targeting mouse VEGFR2 was used as a positive control for down-regulation of the mVEGFR2 gene. The tumor volume was measured as an indication of mVEGFR2 gene silencing.

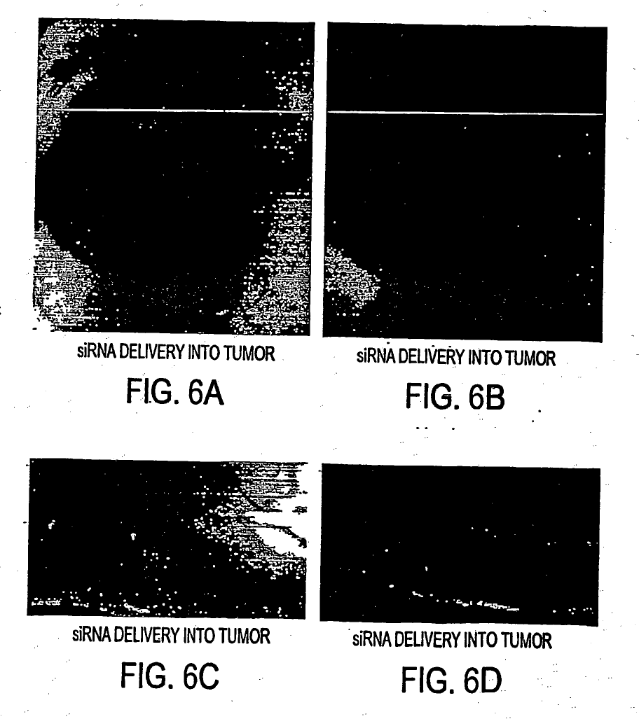

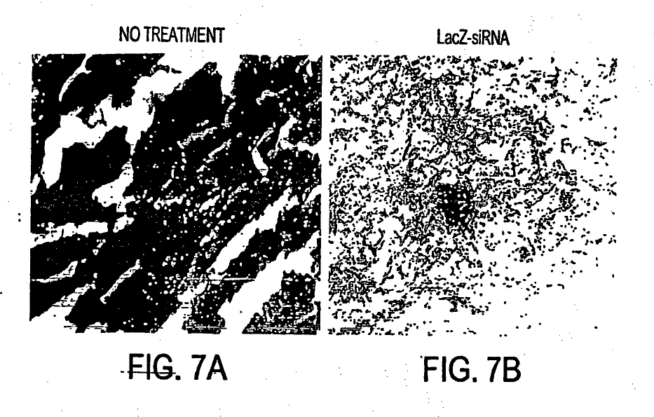

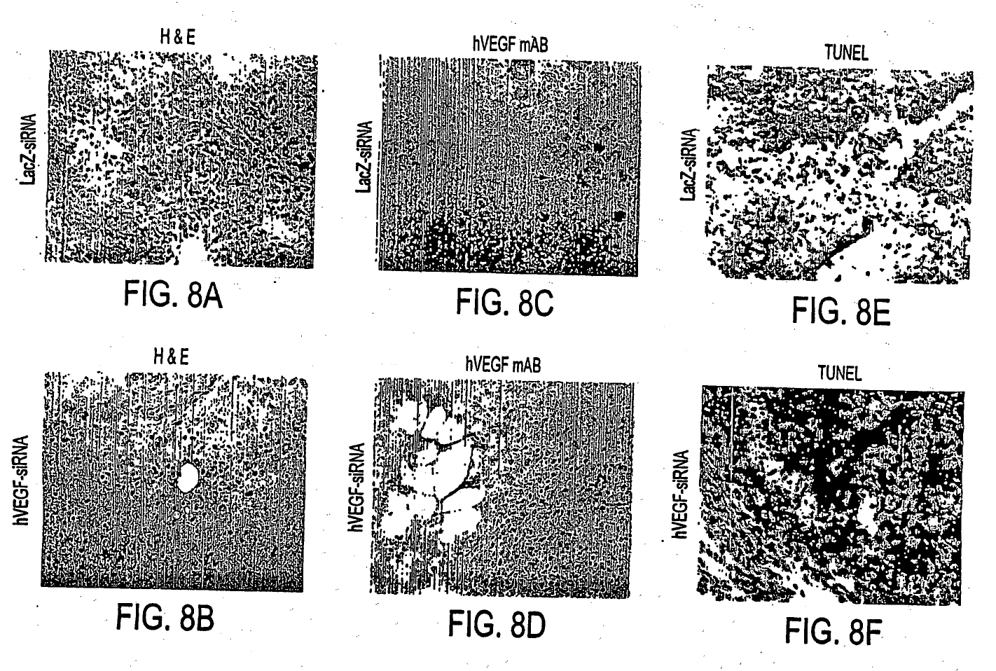

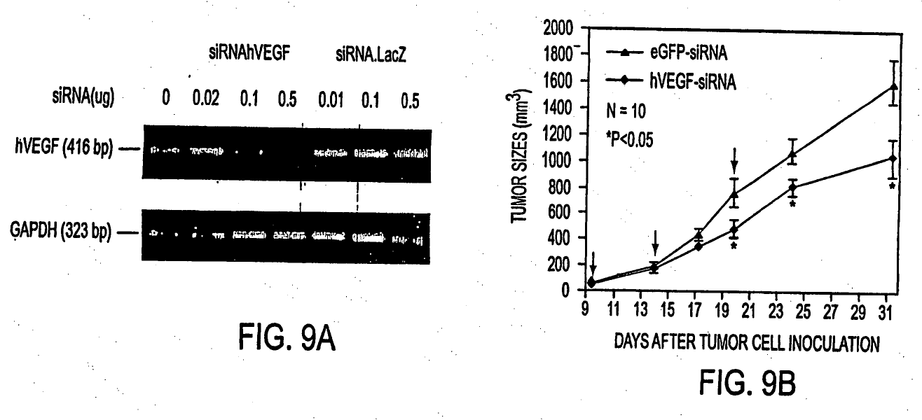

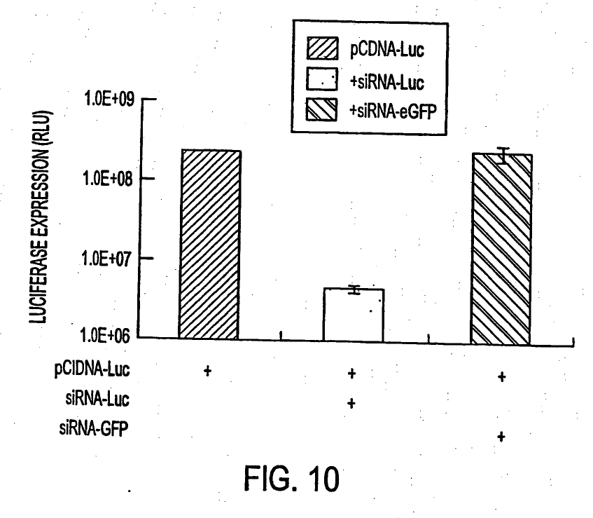

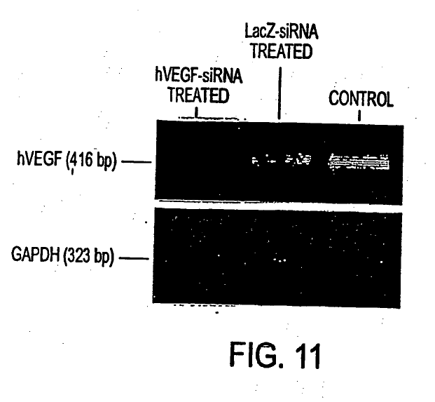

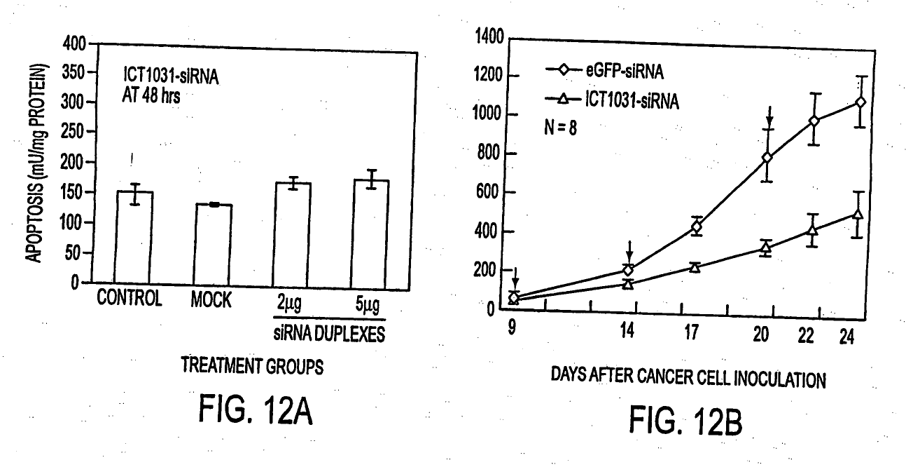

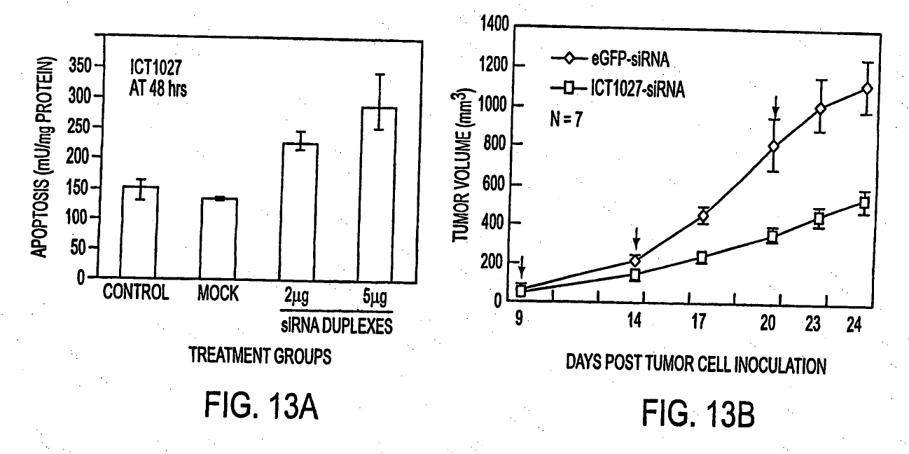

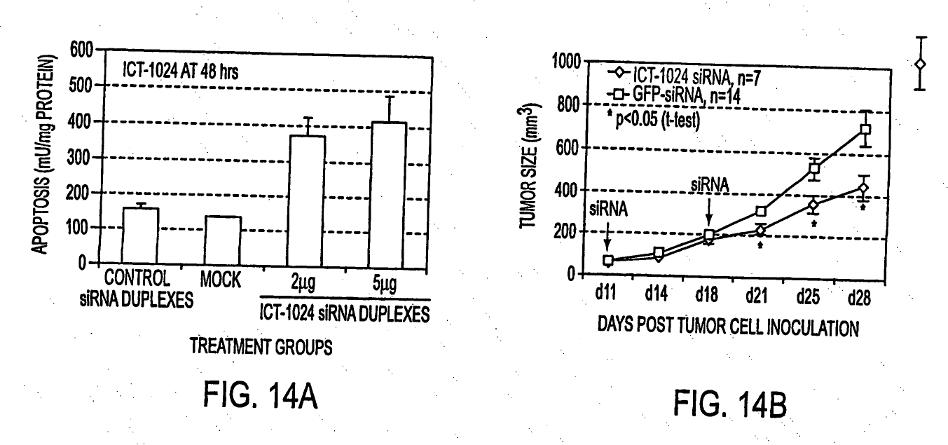

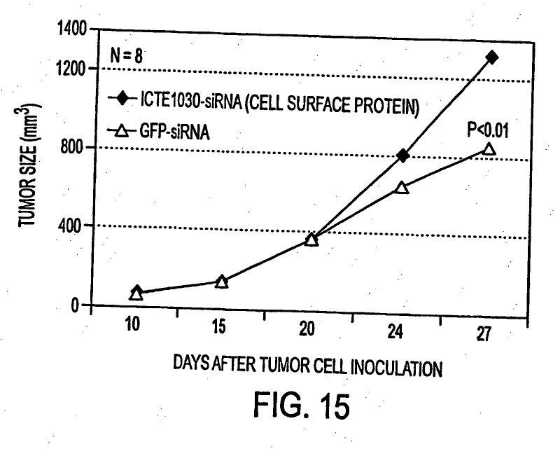

As demonstrated in FIG. 6, tumors treated with dsRNA derived from mVEGFR2 gene grown significantly slower compared to tumors treated with siRNAs derived from LacZ gene. Furthermore, tumors treated with dsRNA derived from mVEGFR2 gene also grown significantly slower compared to tumors treated with mVEGFR2 DNAzyme. On the other hand, tumors treated with siRNAs derived from mVEGFR2 grown at comparable rate with tumors treated with mVEGFR2 DNAzyme, but still significantly slower than tumors treated with siRNAs derived from LacZ gene FIG. 6). Since the LacZ siRNAs treatment did not inhibit tumor growth, it is our conclusion that both mVEGFR2 dsRNA and mVEGFR2 siRNAs specificly silence mVEGFR2 gene in treated MDA-MB-435 tumor and thus slow down tumor growth rate. More biochemistry assays are now being carried out to demonstrate that mVEGFR2 gene in tumor tissue were indeed specificly silenced by dsRNA derived from mVEGFR2 gene.

### EXAMPLE 4

### PolyTran-Mediated RNAi Delivery Inhibits Human MDA-MB-435 Tumor Growth in Mice

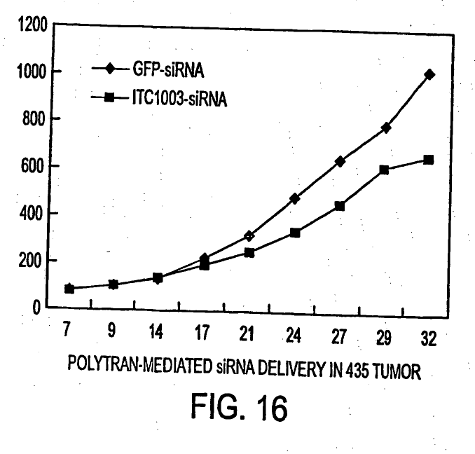

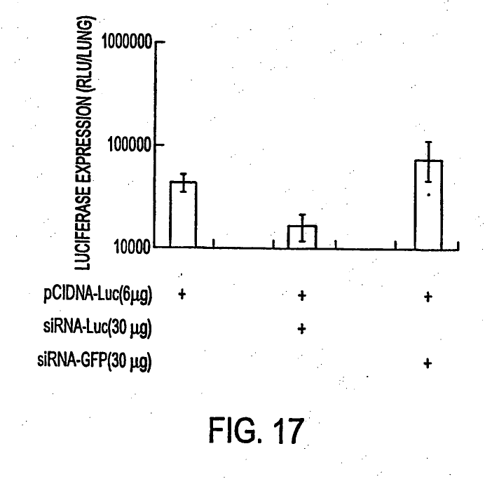

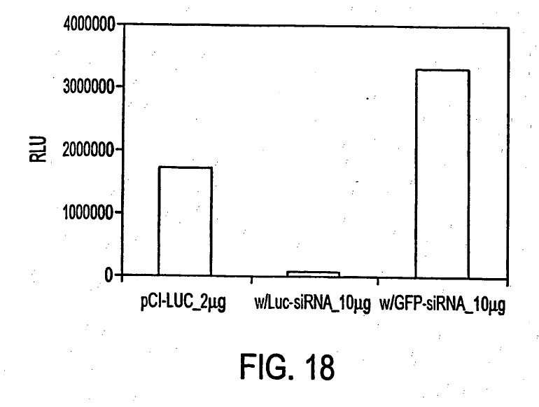

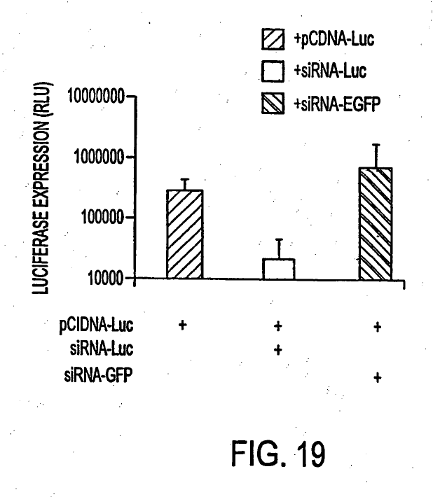

RNAi against targets can be successfully delivered using polymer-mediated delivery as shown by the results in FIG. 16. RNAi directed against the target ICT1003 was delivered to tumor cells using a PolyTran reagent (histidine-lysine copolymer). Briefly, the methods and reagents described in WO01/47496 (which reference is incorporated herein in its entirety) were employed to deliver RNAi to the tumor model described above. GFP-siRNA was used as a control. As shown in FIG. 16, RNAi directed against ICT1003 inhibited tumor growth compared to control. The results shown in FIG. 16 were obtained using the branched reagent HK4b (described in WO01/47496) having the structure [(HK)₄KGK(HK)₄]₄K₃. The skilled artisan will recognize that other HK copolymers may be used and that other cationic polymers known in the art also may be used.

### EXAMPLE 5

### Systemic Delivery of RNAi Using a Targeted Synthetic Vector

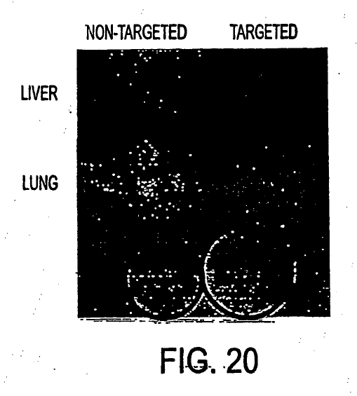

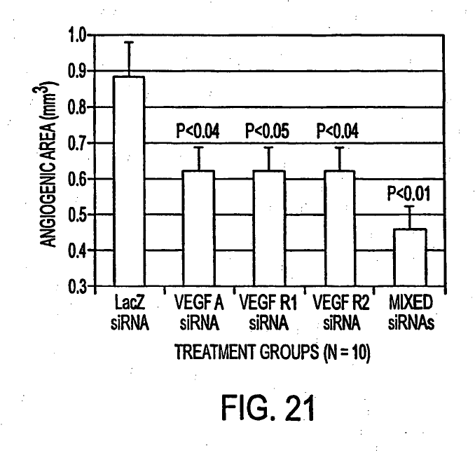

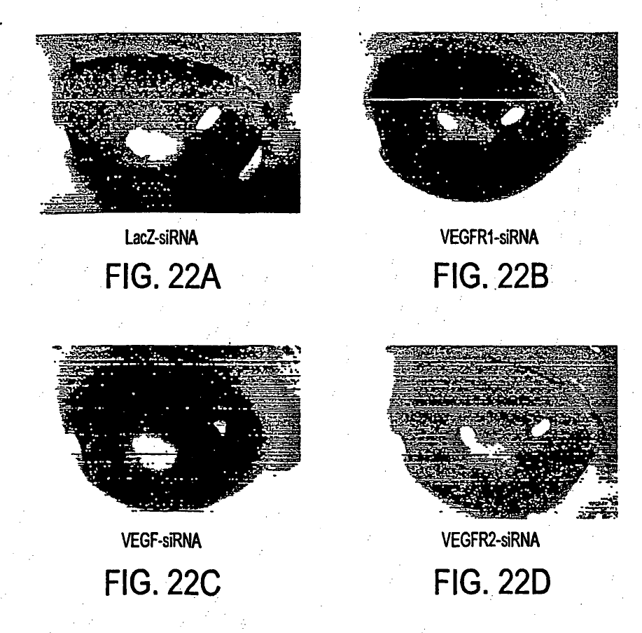

Targeted synthetic vectors of the type described in WO01/49324, which is hereby incorporated by reference in its entirety, may be used for systemic delivery of RNAi. Briefly, a PEI-PEG-RGD (polyethyleneimine-polyethylene glycol-argine-glycine-aspartic acid) synthetic vector was prepared as described, for example, in Examples 53 and 56 of WO01/49324. This vector was used to deliver RNAi systemically via intravenous injection. The results are shown in FIGS. 20-22, which show that anti-VEGF RNAi molecules could successfully delivered using this targeted synthetic vector approach. The skilled artisan will recognize that other targeted synthetic vector molecules known in the art may be used. For example, the vector may have an inner shell made up of a core complex comprising the RNAi and at least one complex forming reagent. The vector also may contain a fusogenic moiety, which may comprise a shell that is anchored to the core complex, or may be incorporated directly into the core complex. The vector may further have an outer shell moiety that stabilizes the vector and reduces nonspecific binding to proteins and cells. The outer shell moiety may comprise a hydrophilic polymer. and/or may be anchored to the fusogenic moiety. The outer shell moiety may be anchored to the core complex. The vector may contain a targeting moiety that enhances binding of the vector to a target tissue and cell population. Suitable targeting moieties are known in the art and are described in detail in WO01/49324.

Other Methods of RNAi Delivery

For certain applications, RNAi may be administered directly as a “naked” reagent with or without electroporation. This can be used, for example, to deliver RNAi molecules and vectors encoding RNAi molecules via direct injections into, for example, tumor tissue and directly into a joint The RNAi may be in a suitable carrier such as, for example, a saline solution or a buffered saline solution.

Target Validation

The ultimate goal of drug target validation is demonstration that a candidate target actually controls the disease. Disease-controlling targets are the high value targets that justify drug discovery. The goal of drug development is products that selectively target key pathways and the key controlling elements of those pathways in order to provide effective therapeutic control of the disease. Validation of such key pathways and elements requires demonstration that addition or subtraction of individual candidate targets controls the disease, i.e. results in a clear increase or decrease of pathology. In vitro cell-based strategies have provided useful information in helping identify and select potential targets. However, the ability of targets to control in vitro cell models associated with disease frequently is not sufficient to prove the target actually controls the disease process, i.e. the complex interactions of multiple cell types that result in disease pathology. Definitive demonstration of disease control by targets can only be obtained by studies of those targets in a true disease model.

The process of target discovery has been greatly accelerated by genomic methods but validation remains a bottlenecks First-generation genomic methods have generated large pools of candidate targets piled up at the validation step. Many approaches are currently being used to study the function of these gene targets and to validate their role in a disease process. Many of these approaches, although having the benefit of being efficient and high throughput, often succeed only at establishing a correlation or association with disease processes rather than determining a controlling role. Newer gene knockdown and forward or inverse genomic approaches have proven useful but these identify genes whose inhibition or mutation may have a disease role, missing potential valuable information from a gene's over-expression. Furthermore, they also employ primarily in vitro-cell-based phenotypes, which do not reflect the complex multi-cellular mechanisms of most diseases, such as tumor angiogenesis, and hence run the risk of missing important targets in adjacent cellular pathways or provide disease associations which are incomplete without the full biological context.

Rapid Definitive Target Validation

The present methods can be used for validating cancer-related drug targets. The methods validate targets directly in animal tumor models by silencing endogenous gene(s) in tumor tissue, and can be used in tandem with methods that involve gene overexpression. See PCT/US02/31554. These methods reduce the need for the costly and slow steps of definitive validation, such as gene cloning and sequencing, generation of proteins and antibodies or transgenic animals. The combination of these two methods vastly accelerates the process, and most importantly rapidly eliminates weaker targets. Moreover, results obtained by the methods provide clear and definitive evidence that targets actually control the disease, the key validation needed to proceed to the costly steps of drug discovery. The methods can be used to complete the validation of any candidate targets such as those generated from cell culture, model organisms, transgenic animals, etc.

Target Discovery: Capturing Targets Missed in Preliminary Validation

Another consideration is that, unfortunately, many high value disease-controlling targets may be lost when in vitro or disease-association methods are employed as the first “filter” in target discovery and validation. Many disease-controller targets may only be found in the context of the entire disease model. For example, targets controlling angiogenesis of tumors will only be found at the conjunction of tumors and blood vessels. In the case of tumors, certain valuable targets may only be discovered by studying the in vivo biological system containing assembly of tumor and surrounding tissues.

High Throughput Target Discovery Solutions

We have also proposed solutions to the challenge of discovering disease controller targets. The solution is to scale-up the basic approach by applying it to screen larger sets of gene targets in a higher throughput operation. By scaling the method to processing multiple candidate genes in animal tumor models, this approach can provide the opportunity to skip, in many cases, preliminary functional validation methods.

Tumor Target Elimination

The present methods, alone or in combination with the methods described in PCT/US02/31554, permit candidate targets to be rapidly tested for their capacity to control tumor growth. Those candidates showing only weak or negligible control of tumor growth can be eliminated from consideration in favor of those that have a strong effect on tumor growth. These Tumor Target Discrimination Methods rapidly discriminates targets into three categories: those enhancing tumor growth, those with little effect on tumor growth, and those inhibiting tumor growth.

### REFERENCES

1. Lu P. Y. et al., Keystone Symposia, Molecular Targets for Cancer Therapy, (20030, p 219.
2. Xu J. et al., Gene Suppression (2003).
3. Lu, Patrick et al., (2002), Cancer Gene Therapy, Vol. 10, Supplement 1, 011.
4. Lu, Patrick Y et al., (2003), Current Opinion in Molecular Therapeutics, 5(3):225-234.
5. Cogoni C. et al., (2000), Genes Dev 10: 638-643.
6. Gumu T., (2000), Nature 404: 804-808.
7. Hammond S M et al., (2001), Nature Rev Gen 2: 110-119.
8. Napoli C et al., (1990), Plant Cell 2: 279-289.
9. Jorgensen R A et al., (1996), Plant Mol Biol, 31: 957-973.
10. Ingelbrecht I et al., (1994), Proc Natl Acad Sci USA, 91: 10502-10506.
11. Cogoni C et al, EMBO J, 15:3153-3163.
12. Palauqui J C et al., (1998), EMBO J, 16: 4738-4745.
13. Guo S et al., (1995), Cell, 81: 611-620.
14. Fire A et al., (1998), Nature 391: 806-811.
15. Timmons L. et al., (1998), Nature 395: 854.
16. Timmons L et al., (2001), Gene 263:103-112.
17. Hunter C P, (2000), Current Biology 10: R137-R140.
18. Tabara H et al., (1998), Science 282: 430-431.
19. Kamath R S et al., (2000), Genome Biology 2: 2.1-2.10.
20. Grishok A et al., (2000), Science 287: 2494-2497.
21. Sharp P A et al., (2000), Science 287: 2431-2433.
22. Sharp P A, (2001), Genes Dev 15: 485-490.
23. Kennerdell J R et al., (1998). Cell 95: 1017-1026.
24. Kennerdell J R et al., (2000), Nature Biotech 18: 896-898.
25. Dzitoyeva S et al., (2001), Mol Psychiatry 6(6):665-670.
26. Worby C A et al., (2001), Sci STKE Aug. 14, 2001(95):PL1.
27. Schmid A et al., (2002), Trends Neurosci 25(2):71-74.)
28. Hamilton A. J. et al., (1999), Science 286: 950-952.
29. Hammond S et al., (2000), Nature, 404: 293-298.
30. Zamore P D et al., (2000), Cell 101: 25-33.
31. Hutvagner G et al., (2002), Curr Opin Genetics & Development 12:225-232.
32. Bernstein E et al., (2001), Nature 409:363-366.
33. Nykanen A et al., (2001), Cell 107:309-321.
34. Lipardi C et al., (2001), Cell 107:297-307.
35. Ketting R F et al., (1999), Cell 99: 133-141.
36. Grishok A et al., (2001), Cell 106:2334.
37. Hutvagner G et al., (2001), Science 293(5531):834-838.
38. Ketting R F et al., (2001), Genes Dev 15(20):2654-2659.
39. Lagos-Quintana M et al., (2001), Science 294:953-858.
40. Lau N C et al., (2001), Science 294:858-862.
41. Lee R C et al., (2001), Science 294:862-864.
42. Ruvkun G., (2001), Science 294:797-799.
43. Manche L et al., (1992), Mol Cell Biol. 12:5238-5248.
44. Minks M A et al., (1979), J. Biol. Chem. 254:10180-10183.
45. Yang S et al., (2001), Mol. Cell. Biol. 21(22):7807-7816.
46. Paddison P J et al., (2002), Proc. Natl. Acad. Sci. USA 99(3):1443-1448.
47. Elbashir S M et al., (2001), Genes Dev 15(2):188-200.
48. Elbashir S M et al., (2001), Nature 411: 494-498.
49. Caplen N J et al., (2001), Proc. Natl. Acad. Sci. USA 98: 9746-9747.
50. Holen T et al., (2002), Nucleic Acids Research 30(8):1757-1766.
51. Elbashir S M et al., (2001), EMBO J 20: 6877-6888.
52. Jarvis R A et al., (2001), TechNotes 8(5): 3-5.
53. Brown D et al., (2002), TechNotes 9(1): 3-5.
54. Brummelkamp T R et al., (2002), Science 296:550-553.
55. Lee N S et al., (2002), Nature Biotechnol. 20:500-505.
56. Miyagishi M et al., (2002), Nature Biotechnol. 20:497-500.
57. Paddison P J et al., (2002), Genes & Dev. 16:948-958.
58. Paul C P et al., (2002), Nature Biotechnol. 20:505-508.
59. Sui G et al., (2002), Proc. Natl. Acad. Sci. USA 99(6):5515-5520.
60. Yu J-Y et al., (2002), Proc. Natl. Acad. Sci. USA 99(9):6047-6052.
61. McCaffrey, A P. et al., (2002), Nature, Vol. 418, July, 2002.

What is claimed is:

1. A method for down regulating a pre-selected endogenous gene in a mammal, comprising administering to a tissue of said mammal a composition comprising a double-stranded RNA molecule wherein said RNA molecule specifically reduces or inhibits expression of said endogenous gene.

2. The method according to claim 1, wherein said RNA molecule is a small interfering RNA or a long double stranded RNA.

3. The method according to claim 2, wherein said RNA molecule is a small interfering RNA molecule having a length of about 21-23 bp.

4. The method according to claim 2, wherein said RNA molecule is a long double stranded RNA having a length of about 100-800 bp.

5. The method according to any preceding claim wherein said composition is administered directly to a tissue of said mammal.

6. The method according to claim 5, wherein administration is via injection into a tumor in said mammal or into a joint in said mammal.

7. The method according to any preceding claim wherein said composition further comprises a polymeric carrier that enhances delivery of said RNA molecule to said tissue of said mammal.

8. The method according to claim 7 wherein said polymeric carrier comprises a cationic polymer that binds to said RNA molecule.

9. The method according to claim 8 wherein said cationic polymer is an amino acid copolymer.

10. The method according to claim 9 wherein said polymer comprises histidine and lysine residues.

11. The method according to claim 10 wherein said polymer is a branched polymer.

12. The method according to any of claims 1-5 wherein said composition comprises a targeted synthetic vector that enhances delivery of said RNA molecule to said tissue of said mammal.

13. The method according to claim 12, wherein said vector comprises a cationic polymer, a hydrophilic polymer, and a targeting ligand.

14. The method according to claim 12, wherein said cationic polymer is a polyethyleneimine.

15. The method according to claim 12, wherein said hydrophilic polymer is a polyethyleneglycol.

16. The method according to claim 15, wherein said targeting ligand is a peptide comprising an RGD sequence.

17. The method according to any preceding claim wherein a pulsed electric field is applied to said tissue substantially contemporaneously with said composition.

18. The method according to any preceding claim wherein said endogenous gene is a mutated endogenous gene.

19. The method according to claim 18 wherein at least one mutation in said mutated gene is in a coding or regulatory region of said gene.

20. The method according to claim 17, further comprising substantially contemporaneously applying a second electric pulse to said tissue.

21. A method for down regulating a pre-selected endogenous gene in a mammal, comprising administering to a tissue of said mammal a vector composition wherein said vector encodes an RNA transcript operatively coupled to a regulatory sequence that controls transcription of said transcript,
and wherein said transcript can form a double stranded RNA molecule in said tissue that specifically reduces or inhibits expression of said endogenous gene.

22. The method according to claim 21, wherein said vector is a viral vector or a plasmid, cosmid or bacteriophage vector.

23. The method according to any preceding claim, wherein said endogenous gene is selected from the group consisting of cancer causing genes, growth factor genes, angiogenesis factor genes, protease genes, protein serine/threonine kinase genes, protein tyrosine kinase genes, protein serine/threonine phosphatase genes, protein tyrosine phosphatase genes, receptor genes, matrix protein genes, cytokine genes, growth hormone genes, and transcription factor genes.

24. The method according to claim 21, wherein said regulatory sequence comprises a promoter.

25. The method according to claim 24 wherein said promoter is a tissue-selective promoter.

26. The method according to claim 25 wherein said tissue-selective promoter is a skin-selective promoter or a tumor selective promoter.

27. The method according to claim 24, wherein said promoter is selected from the group consisting of CMV, RSV LTR, MPSV LTR, SV40, AFP, ALA, OC and keratin specific promoters.

28. The method according to claim 17, wherein said electric pulse comprises a square wave pulse of at least 50 V that is applied to said tissue for between about 10 and about 20 minutes.

29. The method according to claim 28, wherein said electric pulse is monopolar, bipolar or of multiple polarity.

30. The method according to claim 17 wherein said electric pulse comprises an exponential decay pulse of 120 V that is applied to said tissue for between about 10 and about 20 minutes.

31. The method according to claim 17, wherein said electric pulse is applied via an electrode selected from the group consisting of a caliper electrode, a meander electrode, a needle electrode, a micro needle array electrode, a micropatch electrode, a ring electrode, and combinations thereof.

32. The method according to claim 31 wherein said electrode is a caliper electrode having an area of about 1 cm².

33. The method of claim 32 wherein the caliper electrode is applied to a skin fold having a thickness of about 1 mm to about 6 mm.

34. A method for treating a disease in a mammal associated with undesirable expression of a preselected endogenous gene, comprising applying a nucleic acid composition to a tissue of said mammal and substantially contemporaneously applying a pulsed electric field to said tissue, wherein said nucleic acid composition is capable of reducing expression of the endogenous gene in said tissue.

35. The method according to claim 34, wherein said disease is cancer or a precancerous growth.

36. The method according to claim 34, wherein said tissue is a breast tissue, colon tissue, a prostate tissue, a lung tissue or an ovarian tissue.

37. The method according to claim 34, wherein said nucleic acid composition comprises a small interfering RNA, a long double stranded RNA, or a polynucleotide molecule that encodes an RNA transcript that can form a substantially double stranded RNA molecule.

38. The method according to claim 37, wherein said RNA molecule is a small interfering RNA molecule having a length of about 21-23 bp.

39. The method according to claim 37, wherein said RNA molecule is a long double stranded RNA having a length of about 100-800 bp.

40. The method according to claim 39, wherein said RNA has a length of about one hundred base pairs or less.

41. The method according to claim 34, wherein said nucleic acid composition is a vector capable of encoding an siRNA or an RNAi, and wherein said vector is a plasmid, cosmid, bacteriophage, or viral vector.

42. The method according to claim 41, wherein said vector is a retroviral or adenoviral vector.

43. The method according to any preceding claim, wherein said mammal is a human.

44. The method according to claim 34, wherein said preselected endogenous gene is selected from the group consisting of cancer causing genes, growth factor genes, angiogenesis factor-genes, protease genes, protein serine/threonine kinase genes, protein tyrosine kinase genes, protein serine/threonine phosphatase genes, protein tyrosine phosphatase genes, receptor genes, matrix protein genes, cytokine genes, growth hormone genes, and transcription factor genes.

45. The method according to claim 34, wherein said gene is selected from the group consisting of VEGF, VEGF-R, VEGF-R2, VEGF121, VEGF165, VEGF189, and VEGF206.
